(19) United States
(12) Patent Application Publication
(10) Pub. No.: US 2006/0134716 A1
(43) Pub. Date: Jun. 22, 2006

(54) Electrospun enzyme-nanocomposite biosensing material

(76) Inventor: Pelagia-Irene Gouma, Port Jefferson, NY (US)

Correspondence Address: DILWORTH & BARRESE, LLP, 333 EARLE OVINGTON BLVD., UNIONDALE, NY, 11553, US

(21) Appl. No.: 11/304,192

(22) Filed: Dec. 15, 2005

Related U.S. Application Data
(60) Provisional application No. 60/636,463, filed on Dec. 16, 2004.

(51) Int. Cl. C12Q 1/34

(52) U.S. Cl. 435/18

(57) ABSTRACT

The present invention provides biosensing material comprising polymer-enzyme nanocomposite fibers. A biosensor comprising such material may be obtained through an electrospinning process to yield a nonwoven mat, which retains enzyme activity. The large amount of available surface area obtained by the methods of the present invention provides unusually high sensitivity and fast response time in sensing applications. Also provided is a biosensing material for monitoring the concentration of an analyte present in a sample, such as urea. The biosensing material contains nanocomposite fibers of an enzyme, such as urease and at least one polymer produced through an electrospinning process. If desired, the enzyme may be encapsulated inside metal oxide semiconductor thin films. A method for preparing the biosensing material through an electrospinning technique is also provided.

### CROSS REFERENCES

This Application claims the benefit of U.S. Provisional Application No. 60/636,463 filed Dec. 16, 2004, the disclosure of which is incorporated herein by reference.

### FIELD OF THE INVENTION

The present invention relates to biosensing material comprising polymer-enzyme nanocomposite fibers and an electrospinning process for producing the same.

### BACKGROUND OF THE INVENTION

Enzymes are nature's most specific and selective catalysts and many of them have been identified as precise bio-recognition molecules applicable in the sensing field. Enzymes have been known since the early 1960's to be useful tools for detecting the presence of chemical species. See Rogers, K. R., (1995), Biosensors Bioelectronics, 10: 533. Biosensors have been used in the determination of concentrations of various analytes in fluids for more than three decades. Biosensors can be defined as a device that converts biological signal into an electrical output with the detection mechanism utilizing the biological system directly. Of particular interest has been the measurement of blood, glucose, creatinine, creatine and cholesterol. See U.S. patent application Ser. No. 10/861,670.

Generally all enzymatic biosensors function by one of two methods. The enzyme either converts an undetectable compound of interest into another or series of compounds, which can be detected with a chemical-based sensor or the enzyme is inhibited by the presence of the compound of interest and the enzyme inhibition is linked to a measurable quantity. See U.S. Pat. No. 6,291,200, which is incorporated by reference as if fully set forth.

Enzymatic biosensors have been designed to detect a variety of different compounds such as glucose, creatinine, urea, and cholinesterase inhibitors. See Parente, A. H., Marques, E. T. Jr., (1992), Appl. Biochem. Biotechnol. 37, 3, 267; Yang, S., Atanasov, P., Wilkins, E., Ann., (1995), Biomed. Eng., 23, 6, 833. U.S. Pat. No. 5,858,186 describes a urea-based biosensor in which substrate hydrolysis is monitored with a pH electrode. U.S. Pat. Nos. 5,945,343 and 5,958,786 describe enzyme-based sensors in which a fluorophere is immobilized in a first polymer layer and an enzyme is separately immobilized in a second polymer layer. The fluorophere layer fluoresces in the presence of ammonia, which is enzymatically produced from urea and creatinine. In addition, U.S. Pat. No. 4,324,858 describes the immobilization of cholinesterase within a porous, dry material for the colormetric detection of organophosphorus pesticides and nerve agents. U.S. Pat. No. 4,525,704 describes the use of cholinesterases and electrical currents in detecting toxic gases.

Independent of the use thereof, enzyme-based biosensors are often limited in practical application by a number of factors. For example, the process of immobilizing the enzymes using highly specialized synthesis protocols is often expensive and time consuming. Moreover, the sensor often requires specialized electrical equipment to be used in conjunction with the immobilized enzyme, such as a pH meter or an oxygen electrode. See Turner, A. P. F., (1989), Sensors Actuators, 17: 433. The shelf-life, thermal stability, and reusability of enzymatic sensors is often problematic for practical application of the technology. Also, many enzyme-based sensors do not exhibit sufficient sensitivity toward the target compound to monitor the compound over a relevant concentration range. See Evtugyn, G. A., Budnikov, H. C., Kolskaya, (1998), Talanta, 46: 465 as cited in U.S. Pat. No. 6,291,200.

Importantly, the greatest obstacle preventing a large scaled production of enzyme-based sensors is a loss of enzyme activity in even slightly non-biocompatible environments. Some of the stringent requirements to retain enzyme stability are to maintain pH values between 6 and 9, and to maintain an absence of covalent interactions with the medium. As mentioned above, urease is one example of an enzyme that is identified as a precise bio-recognition molecule applicable in the sensing field. Urease acts as a catalyst in the hydrolysis of urea to form carbon dioxide and ammonia; urease increases the hydrolysis reaction by as much as 10¹⁴. Urea is one of the main components of human urine, as the human body digests amines, urea becomes a waste product that builds up in the blood. Abnormal levels of urea in the blood and urine indicate liver function problems. Therefore, urease has found a wide range of applications in the medical field for detoxifying blood in kidney machines. See Qin Y., Carbral J. M. S., “Properties and Applications of Urease”, Biocatalysis and Biotransformation, Vol. 20, pp. 1-14, (2002).

Several technologies proposed for urea detection use immobilized urease. These techniques include immobilizing the urease on gelatin beads, porous glass beads, combining a pH-stat method and flow injection analysis, and preparing electroconductive Pan-PBMA homogenous composite films by casting. As stated above, these biosensing techniques only work in a narrow detection range and therefore have only limited use.

With the increasing demand for nanotechnology, electrospinning has become a technique for generating composite nanofibers. Electrospinning is an atomization process of a conducting fluid which exploits the interactions between an electrostatic field and the conducting fluid. When an external electrostatic field is applied to a conducting fluid (e.g., a semi-dilute polymer solution or a polymer melt), a suspended conical droplet is formed, whereby the surface tension of the droplet is in equilibrium with the electric field. Electrostatic atomization occurs when the electrostatic field is strong enough to overcome the surface tension of the liquid. The liquid droplet then becomes unstable and a tiny jet is ejected from the surface of the droplet. As it reaches a grounded target, the material can be collected as an interconnected web containing relatively fine, i.e. small diameter, fibers. The resulting films (or membranes) from these small diameter fibers have very large surface area to volume ratios and small pore sizes. See, e.g., U.S. Pat. No. 6,713,011 which is incorporated by reference as if fully set forth.

Research in the area of sol-gel encapsulation has emerged rapidly throughout the world and it is now well established that a wide range of biomolecules retain their characteristic reactivities and chemical function when they are confined within the process of the sol-gel derived matrix. See Avnir et al., (1994) Chem. Mater., 6:1605; Dave et al., (1994) Anal. Chem., 66:1120A and U.S. patent application Ser. No. 10/698,042 which is incorporated by reference as if fully set forth.

In addition to extending the sol-gel encapsulation process to numerous other enzymes and other proteins, researchers have expanded the types of biomolecular dopants to include antibodies (J. Livage, et al., (1996) Sol-Gel Sci. Technol. 7: 45) cells, (E. J. A. Pope, et al., J. Sol-Gel Sci. Technol. 8:635), and even photosystems (B. C. Dave, et al., (1996) Mat. Res. Soc. Symp. Proc. 435,565).

It is important to emphasize that the biomolecules are physically immobilized and not covalently attached to the inorganic matrix and, therefore, the ability to incorporate biomolecules in the gel requires only that the synthetic conditions do not cause protein aggregation or denaturation. See J. M. Miller, et al., (1996) J. Non-Crystalline Solids 202, 279. In general, this means that the sol should have minimal alcohol content and pH near 7. The inclusion of the biomolecule in the starting sol leads to a “templating” effect where the inorganic network grows around the dopant molecule. For this reason, a larger biomolecule may be immobilized in the matrix while smaller molecules and ions are free to flow through the porous network. Thus, the microstructure of the sol-gel glass may be tailored so that large protein macromolecules are immobilized in the matrix while analytes are free to enter and diffuse through the porous network. Physical entrapment without chemical modification preserves protein structure and functionality and protects the protein from unfolding (denaturation). The unique advantages of sol-gel immobilization include (1) an easy, simple, more universal method as chemical modification is not necessary, (2) increased durability and ruggedness as these materials can be handled without damage to the biomolecules, (3) more flexibility in sensor design as biologically active materials can be prepared as bulk monoliths or as thin films, and (4) increased stability as the biomolecules are physically, chemically, and microbially protected by a glass matrix.

A further advantage of this technique is that liquid nutrient is co-encapsulated with the bioindicator molecule so that the latter can retain its vitality, but the final composition is truly a solid state device and is dry to the touch and the encapsulated materials do not leach from the matrix. Methods to control and modify the pore size have been reported so that analytes that are relatively large can flow through the matrix and interact with the immobilized bioindicator molecule. See U.S. patent application Ser. No. 10/698,042 which is incorporated by reference as if fully set forth.

The large amount of surface area obtained through both methods has the potential to provide unusually high sensitivity and fast response time in sensing applications. See Zheng-Ming Huang, et al. “A Review of Polymer Nanofibers by Electrospinning and Their Applications in Nanocomposites”, Composite Sci. and Tech., Vol. 63, pp. 2223-2253 (2003).

However, the biosensors using glass beads, porous glass beads, chemical field effect transitor with a pH gate, and Silicon dioxide light addressable potentionmetric devices described above are not cost effective, versatile, capable of measuring small quantities, or have a fast enough response time applicable for real time analysis. Therefore, what is needed is a biosensor that is sensitive enough and reacts quickly enough, and is cost effective so as to overcome the shortcomings of the aforementioned devices.

Accordingly, one object of the present invention is to provide a novel urea bioreceptor by immobilizing urease inside a polymer solution.

Another object of the present invention is to provide an electrospinning technique to produce nanocomposite enzyme-polymer fibers that retain enzymatic activity.

Still another object of the present invention is to provide urea biosensing application that utilizes the high surface area to volume ratio produced by the electrospinning technique of the present invention.

In view of the foregoing objectives, the present invention provides biosensors and a process for producing biosensors that overcome the shortcomings of the described biosensors of the prior art.

### SUMMARY OF INVENTION

In accordance with the present invention, it has now been found that an enzyme may be encapsulated into a biosensor through an electrospinning process yielding a nonwoven mat, which retains enzyme activity. The large amount of available surface area obtained by the methods of the present invention provides unusually high sensitivity, improved adsorption rates, and quick response time in sensing applications.

In a first aspect of the invention, a biosensing material for monitoring the concentration of an analyte present in a sample is provided, wherein the biosensing material contains nanocomposite fibers of at least one polymer and at least one enzyme produced through an electrospinning process.

In one embodiment, a biosensing material for monitoring the concentration of urea in a sample is provided, wherein the biosensing material contains nanocomposite fibers of urease and at least one polymer produced through an electrospinning process.

In another embodiment, a biosensing material containing urease is produced through an electrospinning process and the urease is encapsulated inside metal oxide semiconductor thin films.

In yet another embodiment, the biosensing material containing urease may be used in an application such as peritoneal dialysis.

In still another embodiment, the biosensing material containing urease may be used in an application such as hemodialysis.

In another embodiment, the biosensing material containing urease may be used in an application such as the removal of urea from alcoholic beverages.

In yet another embodiment, the biosensing material containing urease may be used in an application such as the production of ammonia and carbon dioxide.

In still another embodiment, a biosensing material is disclosed, comprising polymer-urease nanofibers wherein the polymer-urease nanofibers retain urease activity, for use in an artificial kidney.

In another aspect of the present invention, a method for preparing the biosensing material is provided, wherein the biosensing material, comprising nanocomposite fibers of at least one polymer and at least one enzyme, is prepared by electrospinning a polymer-enzyme solution, the process comprises, establishing an electric field between a polymer-enzyme solution introduction device and a target, injecting the polymer-enzyme solution fluid from a reservoir under the influence of an electric field, forming a jet of the polymer-enzyme solution, stretching the jet to form a continuous fiber, collecting the fibers on a target, and forming nonwoven mats that are characterized by high surface areas and relatively small pore size. If desired, the enzyme may be encapsulated inside metal oxide semiconductor thin films.

### BRIEF DESCRIPTION OF THE FIGURES

FIG. 1 is an image of the electrospinning set up.

FIG. 2 is a graph of the ammonia concentrations vs. time when urea solutions reacted with (1) 0.2 ml of urease in PBS buffer, (2) 0.2 ml 30% urease in buffer/70% PVP in ethanol solution, and (3) 0.1 ml of urease/PVP nanofiber mat.

FIG. 3(a) is a SEM image of polymer-enzyme nanofibers at 1 μm.

FIG. 3(b) is a SEM image of polymer-enzyme nanofibers covered with cubic salt crystals precipitated from the buffer at 200 μm.

FIG. 4 is a graph of nanofiber size distribution based on diameter.

FIG. 5 is a graph of the conversion from ppm of ammonia to mV.

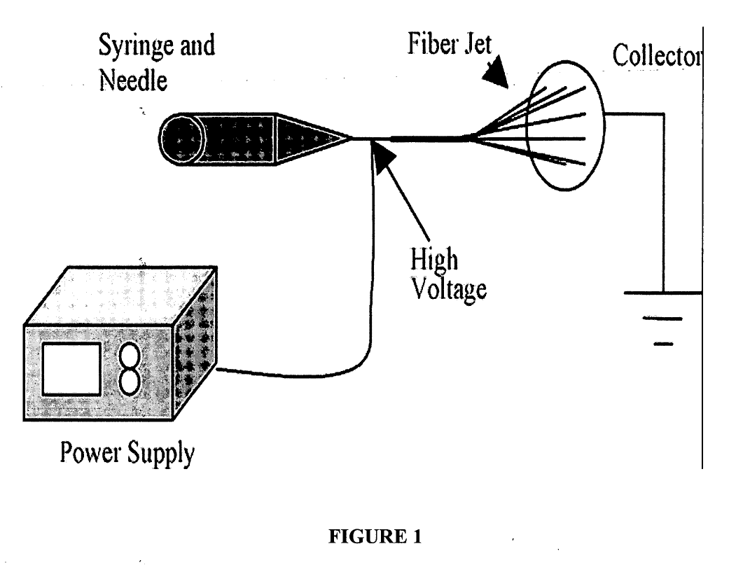

### DETAILED DESCRIPTION

In accordance with the present invention, it has now been found that an enzyme may be encapsulated into a biosensor through an electrospinning process yielding a nonwoven mat, which retains enzyme activity. The large amount of available surface area obtained by the methods of the present invention provides unusually high sensitivity, improved adsorption rates and quick response time in sensing applications.

In electrospinning, the tensile force is generated by the interaction of an applied electric charge carried by the jet rather than by the spindles and reels in conventional spinning. Electrical forces in non-axial directions are also important. By “flow characteristics” (of the polymer solution) is meant the jet formation and jet acceleration of the polymer solution, which exits from the polymer solution introduction device, e.g., the needle tip or glass pipette tip, as well as the directional flow of the jet stream in three-dimensional space. Thus, controlling the flow characteristics can include controlling jet formation, controlling jet acceleration, directing the jet stream to a desired target in three dimensional space, steering the jet stream to different targets during the spinning process or a combination of these.

According to one aspect of the invention, the biosensing material may be prepared by an electrospinning process. In this process a polymer-enzyme solution may be injected from a small nozzle under the influence of an electric field. Applying the external electrostatic field to a conducting fluid (e.g., a charged semi-dilute polymer solution or a charged polymer melt), a suspended conical droplet is formed, whereby the surface tension of the droplet is in equilibrium with the electric field. The build-up of electrostatic charges on the surface of a liquid droplet induces the formation of a jet, which may be subsequently stretched to form a continuous fiber. Before the jet reaches the collecting screen, the solvent may evaporate or solidify. The fibers may be collected on a conductor surface forming nonwoven mats. High surface areas and relatively small pore size characterize the nonwoven mats. The polymer-enzyme solution may be combined with an enzyme in a buffer. The mixture may be about 30% by volume of enzyme in buffer to about 70% polymer. The mixture may be electrospun as soon as introduced to room temperature to form a composite. Enzyme activity may be tested after the completion of the process and compared to pure enzyme in a buffer solution. A known amount of enzyme may be introduced to each solution, and enzyme activity may be observed for twenty minutes.

Once the volume covering the entire surface of the collector can be determined, a quantified volume of the mat can be introduced to the enzyme in a buffer solution. The enzyme retains activity not only inside the polymer-enzyme solution, but also through the harsh process of electrospinning.

The electrospinning process has several advantages including capability of producing fibers in the nanometer diameter range (nanofibers), is driven by electrostatic forces that requires only small amounts of polymer precursors, is a one-step process and does not require further treatment to induce porosity, can produce 1D nanostructures of metal oxides (nanowires), and can be used to incorporate biomolecules into polymer membranes.

In a preferred embodiment of the present invention, the enzyme used to make the biosensor is urease. Urease has a trimer structure and is composed of alpha, beta, and gamma units. Each of these units makes extensive contacts to form a triangle. A flattened sphere of urease has a diameter of about 110 Å and a height of 60 Å. In addition to the three subunits, two nickel atoms are tightly bound to the overall protein and are about 3.5 Å apart and chelated by amino acids. Urease can be used in such applications as peritoneal dialysis. For example, a subject undergoing peritoneal dialysis may use a biosensor wherein the enzyme, urease, is encapsulated into the biosensor through an electrospinning process yielding a nonwoven mat. This process uses the urease to break down urea in the blood and another step to eliminate the ammonia given off.

In addition, urease may be used in hemodialysis. For example a subject undergoing hemodialysis uses a biosensor wherein the enzyme, urease, is encapsulated into the biosensor through an electrospinning process yielding a nonwoven mat. This process uses the urease to break down urea in the blood and another step to eliminate the ammonia given off.

The urease containing biosensor of the present invention may be used in the production of ammonia and/or carbon dioxide. Urease acts as a catalyst in the hydrolysis of urea to ammonia and carbon dioxide. Therefore, a biosensing material, wherein urease is encapsulated into the biosensor through an electrospinning process yielding a nonwoven mat, may be deposited on a substrate, such as a filter, and when a urea containing substance comes in contact with the urea on the substrate, ammonia and/or carbon dioxide is produced.

In addition to the above-mentioned applications, urease containing biosensors of the present invention may be used for treating industrial wastewaters containing urea, wastewater reclamation aboard manned spacecraft, and the analysis of creatinine, arginine, heavy metal ions and other pollutants.

In another aspect of the invention, an artificial kidney is provided comprising a biosensor of the present invention, wherein urease is encapsulated into a biosensor through an electrospinning process yielding a nonwoven mat. The nonwoven mats of the present invention containing composite nanofibers when used in the artificial kidney are successful in retaining enzyme activity, and produce a large surface area characterized by small pore size that provides improved adsorption rate and response time. In other words, smaller concentrations of urea in the fluid being passed through the artificial kidney can be detected and acted upon using the urease containing biosensor of the present invention.

In another aspect of the invention, the biosensors obtained through electrospinning an enzyme-polymer solution to form nanocomposite fibers of enzyme and polymer can be further processed to encapsulate the enzyme inside metal oxide semiconductor thin films. The sol-gel method may be used to encapsulate the enzyme inside metal oxide semiconductor thin films.

In one aspect of the invention, the polymer used in the electrospinning process may include polyvinylpyrrolidone, other polymers known in the art, and mixtures thereof.

In another aspect of the invention, additional or substitute enzymes may be used in the biosensing material. In addition to the urea sensor described above, other sensors are also contemplated, such as a sucrose sensor, maltose sensor, galactose sensor, ethanol sensor, glucose sensor, phenol sensor, catachol sensor, lactic acid sensor, pyruvic acid sensor, uric acid sensor, amino acid sensor, L-glutamine sensor, L-glutamic acid sensor, L-asparagine sensor, L-tyrosine sensor, L-lysine sensor, L-arginine sensor, L-phenylalanine sensor, L-methionine sensor, urea sensor, cholesterol sensor, neutral lipid sensor, phospholipid sensor, monoamine sensor, penicillin sensor, amygdalin sensor, creatinine sensor, phosphate ion sensor, nitrate ion sensor, nitrite ion sensor, sulfate ion sensor, mercury ion sensor, hydrogen peroxide sensor, and mixtures thereof.

In order to illustrate various illustrative embodiments of the present inventions, the following examples are provided.

### EXAMPLE 1

In the following example, a polymer solution was combined with urease in PBS buffer. The mixture was made 30% by volume enzyme in buffer to 70% by volume 4.655×10⁻⁵M polyvinylpyrrolidone (PVP) in ethanol solution. The mixture was electrospun as soon as introduced to room temperature, and formed composite at a voltage of 20 kV and a flow rate of 15 ul/min. The non-woven mats were stored at 4° C. To test urease activity after the process its reactivity was compared to the pure urease in buffer solution. Four urea solutions were prepared, 5 mM, 1.5 mM, 2.0 mM, and 2.5 mM. A known amount of urease was introduced to each solution, and the ammonia concentration given off was observed for 20 minutes. To measure the ammonia concentration, the Thermo Orion ammonia electrode was used. Knowing the volume covering the entire surface of the collector, a quantified volume of the mat was introduced to the urea solutions. Urease: 16,000 units/g×0.0986 g=1577.6 units dissolved in 10 ml of PBS pH of 7.4.

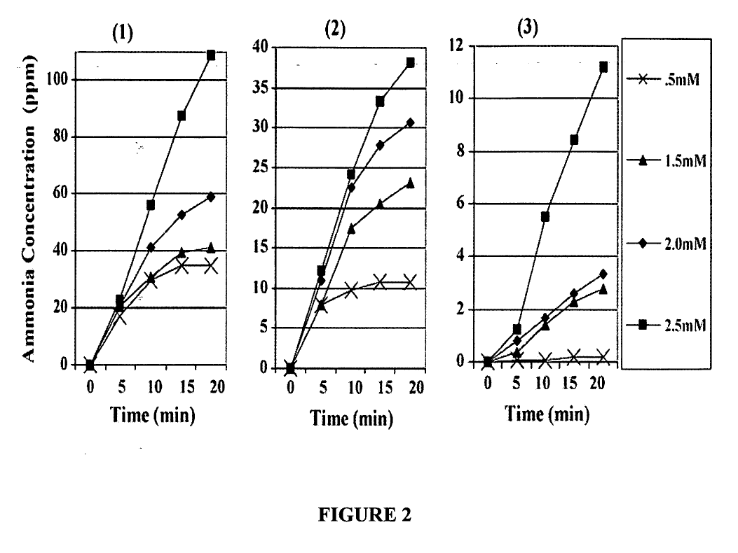

The ammonia concentration observed when electrospun fibers reacted with the urea solutions proved that the enzyme retained activity not only inside the polymer solution, but also through the harsh process of electrospinning. These results are shown in FIG. 2. As shown in FIG. 2, the 30% by volume urease in buffer solution mixed with PVP in ethanol reacted with the urea solutions similarly to the original urease in buffer. Provided that only 30% of the 0.2 ml reacted was enzyme in buffer, the ammonia concentration observed for 0.5 and 2.5 mM urea solutions equaled ⅓ of the ammonia values obtained for pure urease in buffer. The 0.1 ml urease/PVP mat contained only 30% by volume urease in buffer. The ammonia concentration given off when each piece of mat reacted with urea solutions confirmed that the enzyme retained activity through the harsh processing environment of electrospinning.

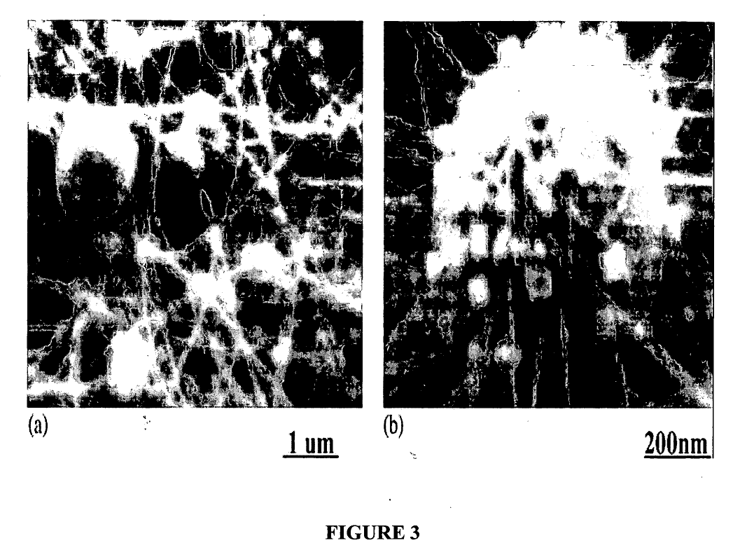

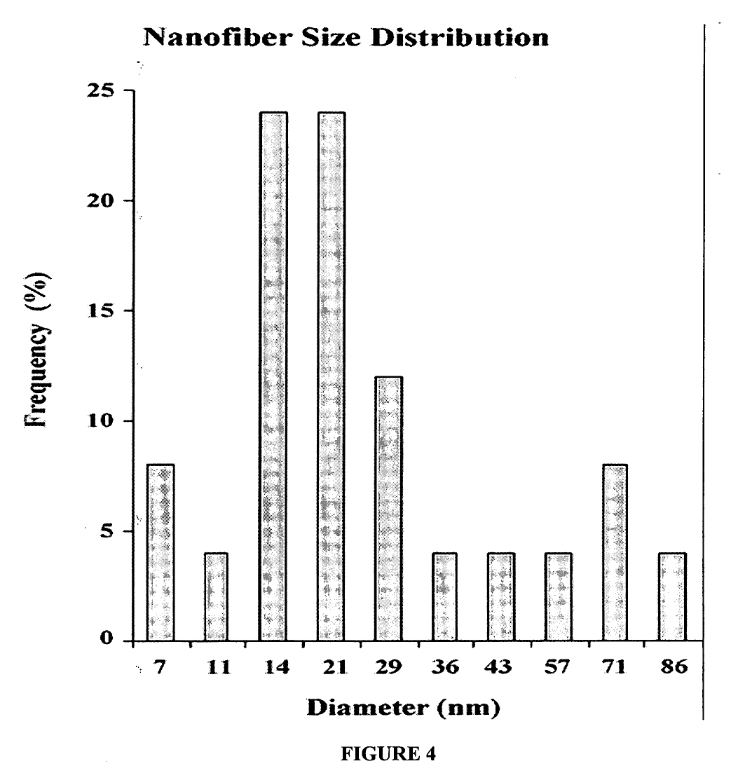

The structure for the electrospun samples was examined using the scanning electron microscope. The fiber diameter varies from about 7 to nm to about 100 nm. The nanofiber diameter size distribution was derived for 25 fibers in a representative area for all samples. The diameter values were obtained from the SEM images as shown in FIG. 3 and the values are presented in FIG. 4. The spherical aggregates of urease molecules varied in diameter from 10 to 800 nm. As seen in FIG. 3b they are covered with cubic salt crystals precipitated from the buffer.

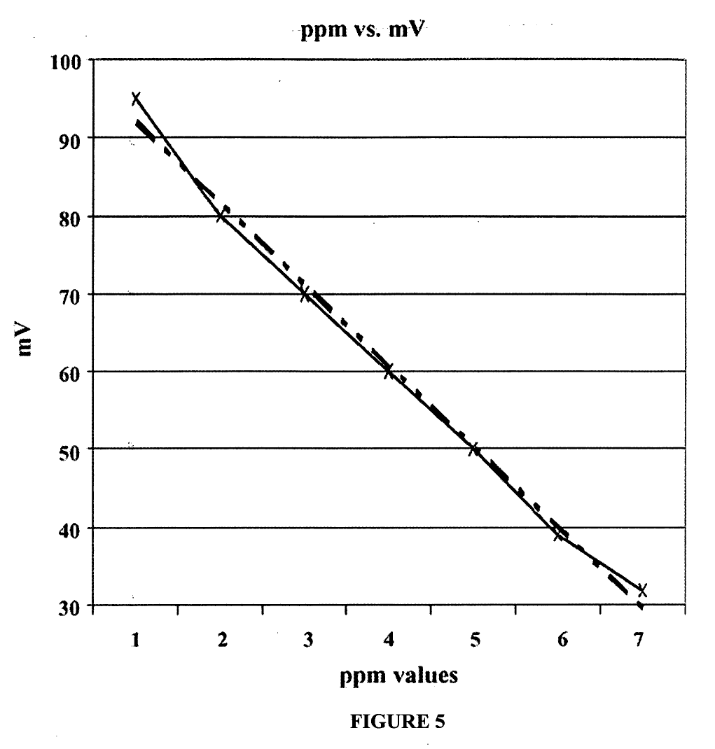

In addition, the proposed material can be employed in potentiometric urea biosensors since the ammonia concentration observed can be easily converted into mV values, through the calibration presented in FIG. 5.

### EXAMPLE 2

The enzyme-polymer solution was prepared by mixing 70% by volume of 4.615×10⁻⁵M polyvinylpyrrolidone (PVP) in ethanol solution (PVP MW=1,300,000), with 30% by volume urease solution with 1577.6 units of urease dissolved in 10 mL of 0.1M PBS buffer. Reactivity measurements were taken for five differently concentrated urea solutions using the Thermo Orion ammonia electrode with the urease/polymer solution before and after electrospinning. The increase in ammonia concentration for both the solution and electrospun fiber mats proved that the enzyme retained activity not only inside the polymer solution, but also through the electrospinning process. For the sol-gel encapsulation, a 0.1M MoO₃ sol-gel was divided into two parts. In both parts, 2 ml of urease solution was added (1577.6 units in water and glycerol). Where one part added it before ultrasonication and the other after one hour of ultrasonication, both of them were then mixed for a total of two hours. Both mixtures retained enzyme activity and acted as catalysts in the hydrolysis.

While the present invention has been described with reference to certain embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out the process of the invention but that the invention will include all embodiments falling within the scope of the appended claims.

What is claimed is:

1. A biosensing material comprising polymer-enzyme nanocomposite fibers wherein the polymer-enzyme nanocomposite fibers retain enzyme activity.

2. The biosensing material of claim 1 produced by an electrospinning process.

3. The biosensing material of claim 2 wherein the enzyme is encapsulated inside metal oxide semiconductor thin films.

4. The biosensing material of claims 1 wherein the enzyme is urease.

5. An artificial kidney comprising the biosensor of claim 4.

6. A method of producing a biosensor comprising:
injecting a solution that comprises at least one polymer, an enzyme and a buffer under the influence of an electric field wherein the build-up of electrostatic charges on a surface of a liquid droplet of the solution induces the formation of a jet;
stretching the induced jet formed by the build-up of electrostatic charges on the surface of the liquid droplet of the solution to form at least one continuous fiber; and
collecting the at least one continuous fiber on a conductor surface to form a nonwoven mat which retains enzyme activity and has a high surface area with a relatively small pore size.

7. The method of claim 6 wherein the solution that comprises at least one polymer, an enzyme and a buffer is produced prior to injecting the solution under the influence of the electric field.

8. The method of claim 6 wherein the enzyme is encapsulated inside a metal oxide semiconductor thin film.

9. The method of claim 6 wherein the solution comprises about 30% by volume of the enzyme and the buffer, the balance of the solution being the polymer.

10. The method according to claim 8 wherein the enzyme is urease.

11. The method according to claim 8 wherein the polymer is polyvinylpyrrolidone.

12. A method of peritoneal dialysis wherein a subject undergoes dialysis using the biosensing material of claim 4.

13. A method of hemodialysis wherein a subject undergoes dialysis using the biosensing material of claim 4.

14. A method of removal of urea from alcoholic beverages wherein the alcoholic beverage is applied to the biosensing material of claim 4 and reacts with the urease to form ammonia and carbon dioxide.

15. A method of analyzing urea concentration in a solution with the biosensing material of claim 4 wherein the solution is reacted with the biosensing material to produce ammonia and the amount of ammonia produced is measured and correlated to the urea concentration in the solution.

16. A method of producing ammonia wherein urea is applied to the biosensing material of claim 4 and reacts with the urease to form ammonia.

17. A method of producing carbon dioxide wherein urea is applied to the biosensing material of claim 4 and reacts with the urease to form carbon dioxide.

18. A method of treating wastewater wherein wastewater comprising urea is applied to the biosensing material of claim 4 and reacts with the urease to form ammonia and carbon dioxide.

19. The method of claim 18 wherein the ammonia produced is removed from the wastewater.

20. The biosensing material of claim 1 wherein the enzyme sensor is selected from the group consisting essentially of a sucrose sensor, maltose sensor, galactose sensor, ethanol sensor, glucose sensor, phenol sensor, catachol sensor, lactic acid sensor, pyruvic acid sensor, uric acid sensor, amino acid sensor, L-glutamine sensor, L-glutamic acid sensor, L-asparagine sensor, L-tyrosine sensor, L-lysine sensor, L-arginine sensor, L-phenylalanine sensor, L-methionine sensor, urea sensor, cholesterol sensor, neutral lipid sensor, phospholipid sensor, monoamine sensor, penicillin sensor, amygdalin sensor, creatinine sensor, phosphate ion sensor, nitrate ion sensor, nitrite ion sensor, sulfate ion sensor, mercury ion sensor, hydrogen peroxide sensor and mixtures thereof.

21. The method of claim 6 wherein the enzyme sensor is selected from the group consisting essentially of a sucrose sensor, maltose sensor, galactose sensor, ethanol sensor, glucose sensor, phenol sensor, catachol sensor, lactic acid sensor, pyruvic acid sensor, uric acid sensor, amino acid sensor, L-glutamine sensor, L-glutamic acid sensor, L-asparagine sensor, L-tyrosine sensor, L-lysine sensor, L-arginine sensor, L-phenylalanine sensor, L-methionine sensor, urea sensor, cholesterol sensor, neutral lipid sensor, phospholipid sensor, monoamine sensor, penicillin sensor, amygdalin sensor, creatinine sensor, phosphate ion sensor, nitrate ion sensor, nitrite ion sensor, sulfate ion sensor, mercury ion sensor, hydrogen peroxide sensor and mixtures thereof.
